(19) United States
(12) Patent Application Publication
(10) Pub. No.: US 2022/0228124 A1
(43) Pub. Date: Jul. 21, 2022

(54) Three-Dimensional Cross-Linked Scaffolds Of Peripheral Blood Plasma And Their Use

(71) Applicant: SANFORD HEALTH, Sioux Falls, SD (US)

(72) Inventors: Pilar DE LA PUENTE, Sioux Falls, SD (US); Somshuvra BHATTACHARYA, Sioux Falls, SD (US); Kristin CALAR, Sioux Falls, SD (US)

(21) Appl. No.: 17/612,142

(22) PCT Filed: Jun. 15, 2019
(86) PCT No.: PCT/US2020/037708

§ 371 Date: Nov. 17, 2021

Related U.S. Application Data
(60) Provisional application No. 62/860,967, filed on Jun. 13, 2019.

(51) Int. Cl. C12N 5/09; C12N 5/00

(52) U.S. Cl. CPC C12N 5/0693; C12N 5/0062; C12N 2500/84; C12N 2533/56; C12N 2500/14; C12N 2513/00

(57) ABSTRACT

The disclosure provides three-dimensional cross-linked scaffolds generated from peripheral blood plasma, and methods for making and using such scaffolds.

### CROSS REFERENCE

This application claims priority to U.S. Provisional Patent Application Ser. No. 62/860,967 filed Jun. 13, 2019, incorporated by reference herein in its entirety.

### FEDERAL FUNDING STATEMENT

This invention was made with government support under Grant No. NIH/NIGMS 5 P20 GM103548-08 awarded by the National Institutes of Health. The government has certain rights in the invention.

### BACKGROUND

Currently available in vitro cell and tissue models for drug screening and other uses do not adequately mimic the in vivo environment of each patient including cellular interactions (cancer, immune, and extracellular matrix), tissue architecture and oxygen availability, directly influencing diffusion capabilities and drug resistance, they rely on exogenous materials to recapitulate the native cellular microenvironment, are not amenable to high-content screening, and their reproducibility and translatability to human clinical trials is very low.

### SUMMARY OF THE DISCLOSURE

In one aspect, the disclosure provides methods comprising:

(a) mixing peripheral blood plasma, with cross-linker and stabilizer to form a mixture; and

(b) incubating the mixture for a time and under conditions to form a three-dimensional cross-linked scaffold.

In one embodiment, the methods comprise pre-mixing the peripheral blood plasma with biological cells to form a pre-mixture, wherein the pre-mixture is mixed with the cross-linker and stabilizer. In various embodiments, the pre-mixing comprises mixing the peripheral blood plasma with the biological cells at room temperature to form the mixture; the peripheral blood plasma comprises peripheral blood plasma obtained from a subject having a tumor or a healthy subject; and/or the biological cells comprise tumor cells, tumor-associated cells, stromal cells or mononuclear cells.

In one embodiment, the cross-linker comprises a cross-linker selected from the group consisting of calcium chloride, thrombin, and factor XIII, or a combination thereof. In another embodiment, the stabilizer is selected from the group consisting of tranexamic acid, aprotinin, epsilon-aminocaproic acid and aminomethylbenzoic acid, or combinations thereof. In a further embodiment, no exogenous polymer is present in the three-dimensional cross-linked scaffold.

In another aspect, the disclosure provides three-dimensional cross-linked scaffolds comprising peripheral blood plasma. In one embodiment, the scaffold further comprises biological cells within the scaffold. In various embodiments, the peripheral blood plasma comprises peripheral blood plasma obtained from a subject having a tumor or healthy subject; and/or the biological cells comprise tumor cells, tumor-associated stromal cells, stromal cells or mononuclear cells. In one embodiment, the scaffold comprises a cross-linker selected from the group consisting of calcium chloride, thrombin, and factor XIII, or a combination thereof. In another embodiment, the scaffold comprises a stabilizer is selected from the group consisting of tranexamic acid, aprotinin, epsilon-aminocaproic acid and aminomethylbenzoic acid, or combinations thereof. In one embodiment, no exogenous polymer is present in the three-dimensional cross-linked scaffold. In another embodiment, the scaffold has an oxygen gradient.

In a further aspect, the disclosure provides methods for use of the three-dimensional cross-linked scaffold of any embodiment or combination of embodiments disclosed herein for any suitable purpose, including but not limited to drug screening, tissue engineering, subject prognosis, cell metabolism, tumor heterogeneity, drug resistance studies, immune and oncology profiling, cell differentiation, toxicology studies, cell fate studies based on exposure to stimuli, inherent cell abnormalities, regenerative medicine, etc. In one embodiment, the methods comprise

(a) contacting the three-dimensional cross-linked scaffold with a test moiety, wherein the test moiety may include, but is not limited to a drug, toxin, hormone, cytokine, small molecule, and/or other stimulus;

(b) culturing the cells of interest within the scaffold; and

(c) determining an effect of the test moiety on the cells of interest.

### DESCRIPTION OF THE FIGURES

FIG. 1(a)-(h). Chemical and physical characterization of human plasma 3D culture model referred as 3DeTME. (a) 3DeTME matrices are formed through the cross-linking of fibrinogen found, naturally in plasma, into fibrin. These matrices can include cells either from cell lines or tissue biopsies. (b) 3DeTME cultures in 96-well plates generate a 3 mm tall gelatinous-like scaffold matrix where media is added on top to overcome drying. (c) A measurement of the time (minutes) to achieve matrix cross-linking using three relevant crosslinking agents of the blood coagulation process including Thrombin (0-5 mg/ml), CaCl₂ (0-5 mg/ml), and Factor XIII (0-6 mg/ml). (d) Stabilization effect studies of preventing fibrin degradation and stability improvement in the scaffold were achieved by testing several chemical antifibrinolytic agents including tranexamic acid (AMCHA) (0-10 mg/ml), Aprotinin (0-550 mg/ml), AECA (0-2.5 mg/ml), and PAMBA (0-2.5 mg/ml). Scaffold stability was studied by measuring each scaffold weight at time 0 and at the conclusion of a 3 week time period. **p<0.001 compared to lack of stabilizer. (e) Representative SEM micrograph of an acellular 3DeTME scaffold cultured for 4 days. Scale bar: 5 μm. (f) Gel stiffness can be chemically or physically controlled recapitulating soft or stiff tissue characteristics measured by atomic force microscopy (AFM). (g) Fibrinogen levels (mg/dL) present in plasma from healthy subjects and cancer patients. (h) Relative protein expression of 3DeTME cultures made of plasma from healthy subjects and cancer patients revealing cytokines involved in key cancer hallmarks including: pro-inflammatory cytokines, cytokines involved in fibrogenesis, cytokines supporting tissue repair/matrix degradation and remodeling, and cytokines promoting cell growth, *p<0.05

FIG. 2(a)-(c). 3DeTME cultures allow cancer cell proliferation. (a) Cell proliferation of BCa cell lines alone or in co-culture with TME in the 3DeTME matrix presented as cell fold of γ₀ for 3 and 7 days. (b) Representative IHC images for Ki67 and caspase 3 staining at days 3 and 7 revealing increased cell survival while unaltered apoptosis, Scale bar=600 μm. (c) Representative confocal images on day 3 and day 7 to monitor proliferation of cancer cells grown within the 3DeTME. Scale bar=1000 μm) revealing cell proliferation over time. **p<0.001, n.s. not significant.

FIG. 3(a)-(b). 3DeTME culture allows high-throughput drug screening in three breast cancer (BCa) cell lines. (a) Results showing the effect of increasing concentrations of Capecitabine, Cyclophosphamide Monohydrate, Docetaxel, Epirubicin Hydrochloride, Methotrexate, Paclitaxel, and Carboplatin on 3 BCa cell lines when grown in 3DeTME cultures on BCa survival and (b) on GR values.

FIG. 4(a)-(e). 3DeTME culture drug metrics correlate better than other in vitro models with clinical data and promote growth of patient biopsy material (fresh or frozen) and recreate therapeutic responses shown in patients in an in vitro environment. (a) Pearson correlation (r) and p significance values of (i) literature 2D IC₅₀ and Clinical Css; (ii) literature 3D IC₅₀ for other 3D models and Clinical Css; (iii) 3DeTME IC₅₀ and Clinical Css. (b) Patient biopsies and blood samples were obtained from cancer patients. Tissue biopsies were either enzymatically digested into single cells or processed into small organoid tissue sections. Both tissue processing methods were grown in 3DeTME cultures made from the matching patient plasma. (c) Cell proliferation in 3DeTME cultures that have been cultured for 3 and 7 days, shown as fold of γ0, either as single cells or small organoids, n.s. not significant. (d) Cell proliferation in 3DeTME cultures that have been cultured for 3 and 7 days, shown as fold of γ0, either as fresh cells or as the same cells subjected to a freeze/thaw cycle (frozen), n.s. not significant. (e) Effect of increasing concentrations of Arimidex (7 days) on cancer cell survival in 3DeTME cultures, highlighting the feasibility of the precision-based capabilities of 3DeTME cultures for the prediction of therapeutic efficacy (*) p<0.05 compared to control, n.s. not significant.

FIG. 5(a)-(d). Development of 3DeTME cultures for recapitulation of physiologically relevant oxygen and tumor-immune interactions. (a) 3DeTME matrices were developed through cross-linking of plasma including cancer cells. PBMCs were added on top of the 3DeTME scaffolds on day 4 of culture and allowed to infiltrate into the scaffold until day 7. (b) Oxygen microsensor (PreSens) and Manual Micromanipulator configuration for O₂ profiling in the Z-direction every 10 μm. (c) Oxygen microsensor was extended delicately and safely with 10 μm profiling accuracy to determine three surface (border between media and 3DeTME matrix) and bottom (bottom of the well) readings. (d) Top to bottom pO₂ levels (kPa) for cell-seeded 3DeTME matrices incubated up to 7 days under 21% and 1.5% O₂.

FIG. 6(a)-(d). Validation of hypoxic phenotype in 3DeTME matrices. (a) Effect of oxygen deprivation on the proliferation of BCa cells grown for 7 days either in 3DeTME physiological or 3DeTME tumorous matrices. (b) HIF-1α expression by cancer cells grown in 3DeTME recapitulating physiological or tumorous pO₂ after 4 days quantified as mean fluorescence intensity (MFI) ratio between AF647-anti-HIF-1α and AF647 isotype control. (c) Mean HIF-1α score indicating the percentage of cancer cells positive for HIF-1α expression after 4 days in 3DeTME physiological and 3DeTME tumorous matrices. (d) ECM expression within 3DeTME physiological and 3DeTME tumorous matrices, quantified as MFI of ECM expression. (**) p<0.001, (*) p<0.05.

FIG. 7(a)-(d). 3DeTME matrices allow the study of lymphocyte infiltration. (a) Quantification of the number of infiltrated lymphocytes into 3DeTME physiological and 3DeTME tumorous matrices by manual gating. Infiltration data shown represents PBMCs average of infiltrated CD3+ T cells, (b) CD3+CD8+ T cells, (c) CD3+CD4+ T cells. (d) Sensitization of BCa cells to cytotoxic CD8+ T cells within 3DeTME matrices. CD8+ infiltration into 3DeTME physiological and 3DeTME tumorous matrices on day 7 after treatment with Durvalumab at 5 μM concentration for the first 4 days. (*) p<0.05.

### DETAILED DESCRIPTION

As used herein, the singular forms “a”, “an” and “the” include plural referents unless the context clearly dictates otherwise. All embodiments of any aspect of the disclosure can be used in combination, unless the context clearly dictates otherwise.

As used herein, “about” means +/31 5% of the recited parameter.

In a first aspect, the disclosure provides methods, comprising:

(a) mixing peripheral blood plasma with cross-linker and stabilizer to form a mixture; and

(b) incubating the mixture for a time and under conditions to form a three-dimensional cross-linked scaffold.

This disclosure provides a tissue-like 3D scaffold that utilizes peripheral plasma as the matrix supporting the recapitulation of cellular interactions, the tissue architecture and oxygen availability without the use of exogenous materials for high-content screening of drug responses for further prediction of precision-based clinical therapeutic efficacy and evaluation of tumor-immunological events. The peripheral plasma sample may be from any suitable subject, including mammals, and particularly human peripheral plasma.

The peripheral blood plasma contains fibrinogen, a plasma glycoprotein involved in the blood coagulation process. The peripheral blood plasma contains pro-inflammatory cytokines, cytokines promoting tissue repair and extracellular matrix remodeling, cytokines promoting cell growth and cytokines involved in fibrogenesis.

The plasma may be freshly prepared, may be thawed from frozen samples, or may be obtained via any other suitable technique. The peripheral blood plasma may be obtained from any suitable source, including but not limited to a patient sample or a healthy subject sample. In one embodiment, peripheral blood plasma is obtained from a subject having a tumor. In this embodiment, the subject may have any type of tumor, including but not limited to an ovarian tumor, a breast tumor, head and neck tumor, lung tumor, colon and rectal tumor, pancreatic tumor, melanoma, kidney cancer, and metastatic tumors. In this embodiment, the resulting three-dimensional cross-linked scaffolds can be used, for example, to generate solid tumors in three-dimensional culture and use them for drug screening, tissue engineering, subject prognosis, cell metabolism, tumor heterogeneity, cell fate studies base on exposure stimuli, drug resistance and toxicology studies, and immune and oncology profiling, or any other suitable purpose. In another embodiment, peripheral blood plasma is obtained from a healthy subject. In this embodiment, the resulting three-dimensional cross-linked scaffolds can be used, for example, to generate healthy tissue in three-dimensional culture and use them for drug screening (such as high throughput drug screening), tissue engineering, subject prognosis, cell metabolism, cellular heterogeneity, cell fate studies base on exposure stimuli, drug resistance and toxicology studies, immune profiling, and precision-based personalized prediction of therapeutic efficacy.

In one embodiment, the method comprises pre-mixing the peripheral blood plasma with biological cells to form a pre-mixture, wherein the pre-mixture is mixed with the cross-linker and stabilizer. The pre-mixing of peripheral blood plasma with biological cells to form a pre-mixture may be carried out under any suitable conditions. In one embodiment, the pre-mixing is carried out at room temperature.

Any suitable biological cells, including but not limited to human cells, may be used as deemed appropriate for an intended use. In various non-limiting embodiments, the peripheral blood plasma comprises peripheral blood from a subject having a tumor or a healthy subject, and the biological cells may comprise, but are not limited to, tumor cells, tumor associated stromal cells, stromal cells or peripheral blood mononuclear cells, and combinations thereof Any suitable tumor cells, tumor-associated stromal cells (tumor-associated fibroblasts, cancer-associated endothelial cells, cancer-associated immune cells, cancer-associated adipocytes, and cancer-associated mesenchymal cells), normal stromal cells (fibroblast, endothelial, immune cells, adipocytes, and mesenchymal cells) or peripheral blood mononuclear cells, and combinations thereof, may be used in this embodiment. In one such embodiment, the biological cells, which may include but are not limited to tumor cells, tumor-associated stromal cells, and/or mononuclear cells, and combinations thereof, are of the same type as the subject's tumor; i.e., if the peripheral blood sample is obtained from a subject having a breast tumor, the tumor, tumor-associated or mononuclear cells for inclusion in the three-dimensional cross-linked scaffold are from the breast tumor or blood. In another non-limiting embodiment, the biological cells are dissociated as single cells for inclusion in the three-dimensional cross-linked scaffold. In another embodiment, the biological cells retain tissue characteristics as organoids for inclusion in the three-dimensional cross-linked scaffold. In another non-limiting embodiment, the biological cells are collected fresh for inclusion in the three-dimensional cross-linked scaffold. In another embodiment, the biological cells may be thawed from frozen specimens for inclusion in the three-dimensional cross-linked scaffold. In another non-limiting embodiment, the peripheral blood sample is obtained from a healthy subject, and the biological cells, including but not limited to stromal and mononuclear cells, stem cells, or combinations thereof, for inclusion in the three-dimensional cross-linked scaffold are from healthy breast tissue or blood. In other embodiments, the tumor cells, tumor-associated stromal cells, stromal cells and mononuclear cells include those of a different tumor type from the subject's tumor. In further embodiments, matched (i.e.: from the same subject) plasma and biological cells can be used, unmatched plasma and biological cells may be used, and matched or unmatched combinations of plasma and biological cells from more than one subject may be used.

The biological cells may be present at any suitable concentration. In one embodiment, the cells are present at between about 10³ and about 10⁷ cells/ml, between about 10³-10⁶ cells/nil, between about 10⁴ and about 10⁷ cells/ml, between about 10⁴ and about 10⁶ cells/ml, about 10³ and about 10⁵ cells/ml, or between about 10⁵ and about 10⁷ cells/nil. In specific embodiments, the cells are present at between about 10⁴ and about 10⁷ cells/ml or between about 10⁵ and about 10⁷ cells/ml.

In various embodiments, the cross-linker comprises a cross-linker selected from the group consisting of calcium chloride, thrombin, and factor XIII, or a combination thereof, and/or the stabilizer is selected from the group consisting of tranexamic acid, aprotinin, epsilon-aminocaproic acid and aminomethylbenzoic acid, or combinations thereof. In a specific embodiment, the cross-linker comprises calcium chloride present at a concentration of between about 0.5 mg/ml and about 5 mg/ml, about 0.5 mg/ml and about 4.5 mg/ml, about 0.5 mg/ml and about 4 mg/ml, about 0.5 mg/ml and about 3.5 mg/ml, about 0.5 mg/ml and about 3 mg/ml, or about 0.5 mg/ml and about 2.5 mg/ml in the mixture (or the resulting cross-linked scaffold). In another specific embodiment, the cross-linker comprises thrombin at a concentration of between about 0.5 mg/ml and about 5 mg/ml, about 1 mg/ml and about 5 mg/ml, about 2 mg/ml and about 5 mg/ml, or about 2.5 mg/ml and about 5 mg/ml in the mixture (or the resulting cross-linked scaffold). In another specific embodiment, the cross-linker comprises activated Factor III at a concentration of between about 0.75 mg/ml and about 6 mg/ml, about 1 mg/ml and about 6 mg/ml, about 1.5 mg/ml and about 6 mg/ml, about 2 mg/ml and about 6 mg/ml, about 2.5 mg/ml and about 6 mg/ml, or about 3 mg/ml and about 6 mg/ml in the mixture (or the resulting cross-linked scaffold). In one specific embodiment, the cross linker comprises calcium chloride, as it provides the fastest cross-linking time and is more readily available than thrombin and factor XIII.

In another embodiment, the stabilizer comprises (i) tranexamic acid present at a concentration of between about 0.5 mg/ml and about 10 mg/ml, about 1 mg/ml and about 10 mg/ml, about 2 mg/ml and about 10 mg/ml, about 2.5 mg/ml and about 10 mg/ml, about 3 mg/ml and about 10 mg/ml, about 3.5 mg/m1 and about 10 mg/ml, about 4 mg/ml and about 10 mg/ml, about 4.5 mg/ml and about 10 mg/ml, or about 5 mg/ml and about 10 mg/ml in the mixture (or the resulting cross-linked scaffold); (ii) aprotinin present at a concentration of between about 50 mg/ml and about 550 mg/ml, about 75 mg/ml and about 550 mg/ml, about 95 mg/ml and about 550 mg/ml, or about 110 mg/ml and about 550 mg/ml in the mixture (or the resulting cross-linked scaffold); (iii) epsilon-aminocaproic acid at a concentration of between about 0.5 mg/ml and about 2.5 mg/ml, about 0.5 mg/ml and about 2 mg/ml, about 0.5 mg/ml and about 1.5 mg/ml, about 0.5 mg/ml and about 1 mg/ml, or about 0.5 mg/ml and about 0.5 mg/ml in the mixture (or the resulting cross-linked scaffold); (iv) aminomethylbenzoic acid at a concentration of between about 0.5 mg/ml and about 2.5 mg/ml, about 0.5 mg/ml and about 2 mg/ml, about 0.5 mg/ml and about 1.5 mg/ml, about 0.5 mg/nil and about 1 mg/ml in the mixture (or the resulting cross-linked scaffold); or (v) combinations thereof. In a specific embodiment, the stabilizer comprises tranexamic acid, which induces a higher weight gain in the matrix when compared to the others.

The plasma, crosslinker, and stabilizer may be mixed in a separate container and then aliquoted into multiple wells for cross-linking as deemed appropriate for an intended use. In various embodiments, the plasma, crosslinker and stabilizer may be aliquoted into microtiter wells (for example, 24-well, 48-well, or 96-well plates), well chambers, or capsules prior to cross-linking.

Any suitable incubating conditions may be used that lead to cross-linking. In one embodiment, the cross-linking incubation is carried out at about room temperature. The incubating can be carried out for any suitable period of time to accomplish the desired amount of cross-linking. In various embodiment, the cross-linking incubating is carried out for between about 5 minutes to about 8 hours, about 5 minutes to about 6 hours, about 5 minutes to about 4 hours, about 5 minutes to about 2 hours, about 30 minutes to about 8 hours, about 30 minutes to about 6 hours, about 30 minutes to about 4 hours, about 30 minutes to about 2 hours; about 1 hour to about 8 hours, about 1 hour to about 6 hours, about 1 hour to about 4 hours, about 1 hour to about 2 hours, about 2 hours to about 8 hours, about 2 hours to about 6 hours or about 2 hours to about 4 hours.

In another embodiment, no exogenous polymer is present in the three-dimensional cross-linked scaffold, which minimizes the manipulation of the natural development microenvironment provided by the scaffolds of the disclosure. In another embodiment, one or more other polymers may be added as appropriate for an intended use, including but not limited to increasing stiffness of the scaffold. In this embodiment, three-dimensional cross-linked scaffolds can recapitulate soft or stiff tissue characteristics.

The peripheral blood plasma may be present in the mixture at any suitable concentration. In various embodiments, the peripheral blood plasma is present in the mixture at a concentration of between about 30% v/v and about 80% v/v, about 30% v/v and about 70% v/v, about 30% v/v and about 60% v/v, or between about 30% v/v and about 50% v/v.

After cross-linking, cell culture media may be added to the scaffold and the scaffolds further incubated for cell growth and any uses, including but not limited to those disclosed herein. Any cell culture medium suitable for the biological cells in the scaffold may be used. The medium may be added to the top of the scaffold, may be added through the wall of the well (i.e.: not directly on top of the 3D culture), or may be added to the scaffold in any other suitable manner. In one embodiment, the peripheral blood plasma is from a subject having a tumor or healthy subject, and the culturing is carried out for a time and under conditions suitable to promote formation of a tumor or healthy tissue within the three-dimensional cross-linked scaffold. In a further embodiment, the methods may comprise adding a second population of cells to the top of the scaffold and culturing the second population of cells on the scaffold. In one non-limiting embodiment, the second population may comprise tumor cells, tumor associated stromal cells, stromal cells or peripheral blood mononuclear cells, i.e., immune cells, including but not limited to T cells, B cells, NK cells, myeloid-derived suppressor cells and monocytes. In this embodiment, the effect on the second population of cells on cells within the scaffold (such as tumor cells, tumor-associated tumor cells, stromal cells, or mononuclear cells of a healthy or solid tumor derived therefrom) can be tested in the presence or absence of test compounds. In one non-limiting embodiment, the second population may comprise stromal cells (i.e.: mesenchymal, endothelial, immune cells including but not limited to T cells, B cells, NK cells, myeloid-derived suppressor cells and monocytes). In this embodiment, the effect on the second population of cells on cells within the scaffold can be tested in the presence or absence of test compounds. In these embodiments, the second population of cells can be used to recreate different tissue-specific cellular niches.

In various embodiments, the methods may comprise modifying the oxygen environment during the mixing and incubating steps. For example, oxygen content may be manipulated by incubating the scaffolds in an oxygen-deprived or oxygen-enriched environment, or by chemically-inducing hypoxia (including but not limited to incorporation of chemicals such as CoCl2). In one non-limiting example, scaffolds prepared in a 21% oxygen environment can include an oxygen partial pressure of 7 kPa vs 0.73 kPa if scaffolds are prepared in a at 1.5% oxygen environment. The bottom of these gels can be 5 kPa for 21 and 0.3 kPa for 0.5% oxygen incubation. Any incubation in between 21% oxygen and 0.5% can be manipulated to generate the desired oxygen level.

In another embodiment, post-cross-linking steps, such as adding cell culture medium, cell proliferation differentiation, and the recited uses, may be carried out at between about room temperature and about 37° C.

In a second aspect, the disclosure provides three-dimensional cross-linked scaffolds made by the method of any embodiment or combination of embodiments of the first aspect of the disclosure.

In a third aspect, the disclosure provides three-dimensional cross-linked scaffolds comprising peripheral blood plasma. The peripheral blood plasma may be obtained from any suitable source, including but not limited to a patient sample or a healthy subject sample. In one embodiment, peripheral blood plasma is obtained from a subject having a tumor. In this embodiment, the subject may have any type of tumor, including but not limited to an ovarian tumor, a breast tumor, head and neck tumor, lung tumor, colon and rectal tumor, pancreatic tumor, melanoma, kidney cancer, or metastatic tumor.

In one embodiment, the scaffold further comprises biological cells within the scaffold. The biological cells may comprise, but are not limited to, tumor cells, tumor associated stromal cells, (tumor-associated fibroblasts, cancer-associated endothelial cells, cancer-associated immune cells, cancer-associated adipocytes, and cancer-associated mesenchymal cells), normal stromal cells (fibroblast, endothelial, immune cells, adipocytes, and mesenchymal cells), mononuclear cells from healthy subjects or from subjects with tumors, such as immune cells including but not limited to T cells, B cells, NK cells, myeloid-derived suppressor cells and monocytes), and combinations thereof. In this embodiment, the resulting three-dimensional cross-linked scaffolds can be used, for example, to generate solid tumors or healthy tissues in three-dimensional culture and use them for drug screening, tissue engineering, subject prognosis, cell metabolism, tumor heterogeneity, cell fate studies base on exposure stimuli, drug resistance and toxicology studies, and immune and oncology profiling any other suitable purpose. In other embodiments, the tumor cells include those of a different tumor type from the subject's tumor.

In one embodiment, the biological cells are present in the scaffold at a concentration between about 10³ cells/ml and about 10⁷ cells/ml, between about 10³-10⁶ cells/ml, between about 10⁴ and about 10⁷ cells/ml, between about 10⁴ and about 10⁶ cells/ml, about 10³ and about 10⁵ cells/ml, or between about 10⁵ and about 10⁷ cells/ml. In specific embodiments, the cells are present at between about 10⁴ and about 10⁷ cells/ml or between about 10⁵ and about 10⁷ cells/ml.

In one embodiment, the three-dimensional cross-linked scaffold comprises a cross-linker selected from the group consisting of calcium chloride, thrombin, and factor XIII, or a combination thereof. In various embodiments, the three-dimensional cross-linked scaffold comprises (i) calcium chloride present at a concentration of between about 0.5 mg/ml and about 5 mg/ml, about 0.5 mg/ml and about 4.5 mg/ml, about 0.5 mg/ml and about 4 mg/ml, about 0.5 mg/ml and about 3.5 mg/ml, about 0.5 mg/ml and about 3 mg/ml, or about 0.5 mg/ml and about 2.5 mg/ml, (ii) thrombin at a concentration of between about 0.5 mg/ml and about 5 mg/ml, about 1 mg/ml and about 5 mg/ml, about 2 mg/ml and about 5 mg/ml, or about 2.5 mg/ml and about 5 mg/ml; (iii)activated Factor III at a concentration of between about 0.75 mg/ml and about 6 mg/ml, about 1 mg/ml and about 6 mg/ml, about 1.5 mg/ml and about 6 mg/ml, about 2 mg/ml and about 6 mg/ml, about 2.5 mg/ml and about 6 mg/ml, or about 3 mg/ml and about 6 mg/ml; or (iv) combinations thereof In one specific embodiment, the cross linker comprises calcium chloride.

In another embodiment, the scaffold comprises a stabilizer. In various embodiments, the stabilizer comprises (i) tranexamic acid present at a concentration of between about 0.5 mg/ml and about 10 mg/ml, about 1 mg/ml and about 10 mg/ml , about 2 mg/ml and about 10 mg/ml, about 2.5 mg/ml and about 10 mg/ml, about 3 mg/ml and about 10 mg/ml, about 3.5 mg/ml and about 10 mg/ml, about 4 mg/ml and about 10 mg/ml, about 4.5 mg/ml and about 10 mg/ml, or about 5 mg/ml and about 10 mg/ml; (ii) aprotinin present at a concentration of between about 50 mg/ml and about 550 mg/ml, about 75 mg/ml and about 550 mg/ml, about 95 mg/ml and about 550 mg/ml, or about 110 mg/ml and about 550 mg/ml; (iii) epsilon-aminocaproic acid at a concentration of between about 0.5 mg/ml and about 2.5 mg/ml, about 0.5 mg/ml and about 2 about 0.5 mg/ml and about 1.5 mg/ml, about 0.5 mg/ml and about 1 mg/ml, or about 0.5 mg/ml and about 0.5 mg/ml; (iv) aminomethylbenzoic acid at a concentration of between about 0.5 mg/ml and about 2.5 mg/ml, about 0.5 mg/ml and about 2 mg/ml, about 0.5 mg/ml and about 1.5 mg/ml, about 0.5 mg/ml and about 1 mg/ml; or (v) combinations thereof. In a specific embodiment, the stabilizer comprises tranexamic acid.

In a further embodiment, no exogenous polymer is present in the three-dimensional cross-linked scaffold. In another embodiment, the peripheral blood plasma is present in the mixture at a concentration of between about 30% v/v and about 80% v/v, about 30% v/v and about 70% v/v, about 30% v/v and about 60% v/v, or between about 30% v/v and about 50% v/v.

In all embodiments disclosed herein, the three-dimensional cross-linked scaffold may be of any suitable thickness. In various embodiments, the three-dimensional cross-linked scaffold has a thickness of between about 100 μm and about 3000 μm, between about 100 μm and about 2500 μm, between about 100 μm and about 2000 μm, between about 100 μm and about 1500 μm, between about 100 μm and about 1000 μm, between about 100 μm and about 900 μm, between about 100 μm and about 800 μm, between about 100 μm and about 700 μm, between about 100 μm and about 600 μm, between about 100 μm and about 500 μm, between about 100 μm and about 400 μm, between about 200 μm and about 1000 μm, between about 200 μm and about 900 μm, between about 200 μm and about 800 μm, between about 200 μm and about 700 μm, between about 200 μm and about 600 μm, between about 200 μm and about 500 μm or between about 200 μm and about 400 μm.

In another embodiment, the three-dimensional cross-linked scaffold has an oxygen gradient or recapitulates different physiologically relevant oxygen values to healthy tissue or tumorous tissue. The oxygen levels may be controlled, for example, by controlling the thickness of the scaffold, by incubating the scaffold in a controlled oxygen environment or by chemical-induction. By way of non-limiting example, in areas of the scaffold with low oxygen, cells do not proliferate but secrete a lot of matrix, making the area stiffer and affecting drug transport, cell motility, and cell migration.

In one embodiment, the scaffold thickness can be modified to modify oxygen levels through the depth of the scaffold. For example, a 1 mm gel can have an oxygen gradient difference top to bottom of 0.4 kPa, a 2 mm tall gel 0.7 kPa, 3 mm gel 2 kPa, and so forth. In other embodiments, scaffolds of a consistent height may be preferred, for example, to limit the use of patient-derived resources, and oxygen content can be manipulated by preparing/incubating the scaffolds in an oxygen deprived incubator or by chemically-inducing hypoxia (including but not limited to incorporation of chemicals such as CoCl2). In one non-limiting example, scaffolds prepared in a 21% oxygen environment can include an oxygen partial pressure of 7 kPa vs 0.73 kPa if scaffolds are prepared in a at 1.5 oxygen environment. The bottom of these gels can be 5 kPa for 21 and 0.3 kPa for 0.5% oxygen incubation. Any incubation in between 21% oxygen and 0.5% can be manipulated to generate the desired oxygen level.

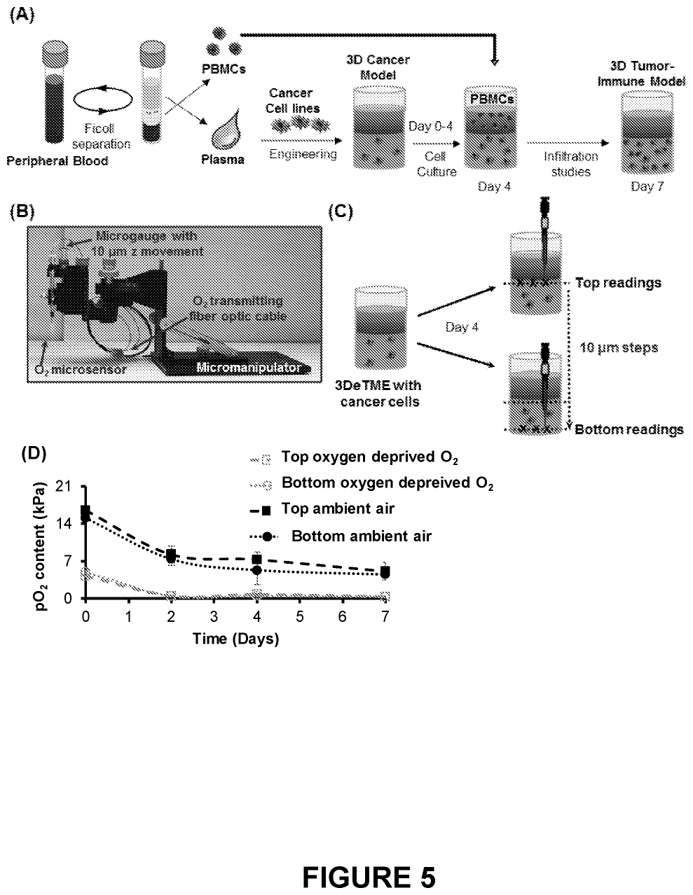

Furthermore, cellular concentration and cell type will affect the oxygen availability. The more cells and more proliferative activity, the less oxygen will be available in the scaffold. Finally, oxygen availability will vary over time if there are cells that consume that oxygen (see FIG. 5D).

In various embodiments, the oxygen partial pressure (pO₂) levels in the scaffold range between about 8.6 kPa and about 1.4 kPa, between about 8.6 kPa and about 2.5 kPa, between about 8.6 kPa and about 3.5 kPa, between about 8.6 kPa and about 4.5 kPa, between about 8.6 kPa and about 5.3 kPa, between about 8.6 kPa and about 5.9 kPa, between about 7.3 kPa and about 5.3 kPa for non-tumor scaffolds. In other embodiments, the oxygen pO₂ levels in the scaffold range between about 1.5 kPa and about 0.2 kPa, between about 1.5 kPa and about 0.3 kPa, between about 1.5 kPa and about 0.7 kPa, between about 1.2 kPa and about 0.2 kPa, between about 1.2 kPa and about 0.3 kPa, between about 1.2 kPa and about 0.7 kPa, or between about 0.7 kPa and about 0.3 kPa for tumor scaffolds.

In another embodiment, a stiffness of the scaffold ranges between about 0.5 kPa to 7 kPa. In one embodiment, a non-tumor scaffold may have a stiffness between about 0.5 kPa to about 7 kPa, between about 0.5 kPa to about 6 kPa, between about 0.5 kPa to about 5 kPa, between about 0.5 kPa to about 4 kPa, between about 0.5 kPa to about 3 kPa, or between about 0.5 kPa to about 2 kPa. In a specific embodiment, a non-tumor scaffold may have a stiffness between about 0.5 kPa to about 2 kPa. In another embodiment, a scaffold comprising tumor cells may have a stiffness between about 0.5 kPa to about 7 kPa, between about 1 kPa to about 6 kPa, between about 1 kPa to about 5 kPa, between about 1 kPa to about 4 kPa, or between about 2 kPa to about 4 kPa, or between about 0.5 kPa to about 2 kPa. In a specific embodiment, scaffold comprising tumor cells may have a stiffness between about 2 kPa to about 4 kPa. Stiffness can be chemically-induced, or may be modified via the cells and oxygen levels.

In another embodiment, the three-dimensional cross-linked scaffolds comprise a porous structure with a network of interconnecting fibrinogen fibers. This embodiment aids, for example, in gas diffusion, nutrient supply, and waste removal through the 3D scaffold. In embodiments in which the scaffolds contain other biological cells, the fibers may further comprise extracellular matrix fibers secreted by the cells, including but not limited to collagen, fibronectin, and laminin. The main regulator of porosity is the fibrinogen content, but porosity can also be modulated with the crosslinkers and other chemical-inducers or by incorporating other proteins (extracellular matrix, such as collagen, laminin, etc). In various embodiments, the porosity is between about 0.5 μm and about 20 μm, between about 1 μm and about 15 μm, between about 1.5 μm and about 10 μm, or between about 2 μm and about 8 μm in diameter. In a specific embodiment, the porosity is between 2 μm and about 8 μm in diameter.

In a fourth aspect, the disclosure provides uses of the three-dimensional cross-linked scaffold of any embodiment of combination of embodiments disclosed herein for any suitable purpose, including but not limited to drug screening, tissue engineering, subject prognosis, cell metabolism, tumor heterogeneity, drug resistance studies, immune and oncology profiling, cell differentiation, toxicology studies, cell fate studies based on exposure to stimuli, inherent cell abnormalities, regenerative medicine, etc. In one embodiment, such use may comprise

(a) contacting the three-dimensional cross-linked scaffold with a test moiety, wherein the test moiety may include, but is not limited to a drug, toxin, hormone, cytokine, small molecule, and/or other stimulus;

(b) culturing the cells of interest within and/or on top the scaffold; and

(c) determining an effect of the test moiety on the cells of interest.

As discussed above, after cross-linking, cell culture media may be added to the scaffold and the scaffolds further incubated for cell growth and any uses, including but not limited to those disclosed herein. Any cell culture medium suitable for the biological cells in the scaffold may be used. The medium may be added to the top of the scaffold, may be added through the wall of the well (i.e.: not directly on top of the 3D culture), or may be added to the scaffold in any other suitable manner. In one embodiment, the peripheral blood plasma is from a subject having a tumor or healthy subject, and the culturing is carried out for a time and under conditions suitable to promote formation of a tumor or healthy tissue within the three-dimensional cross-linked scaffold. In a further embodiment, the methods may comprise adding a second population of cells to the top of the scaffold and culturing the second population of cells on the scaffold. In one non-limiting embodiment, the second population may comprise tumor cells, tumor associated stromal cells, stromal cells or peripheral blood mononuclear cells, i.e immune cells including but not limited to T cells, B cells, NK cells, myeloid-derived suppressor cells and monocytes. In this embodiment, the effect on the second population of cells on cells within the scaffold (such as tumor cells, tumor-associated tumor cells, stromal cells, or mononuclear cells of a healthy or solid tumor derived therefrom) can be tested in the presence or absence of test compounds. In one non-limiting embodiment, the second population may comprise stromal cells (i.e.: mesenchymal, endothelial, immune cells including but not limited to T cells, B cells, NK cells, myeloid-derived suppressor cells and monocytes). In this embodiment, the effect on the second population of cells on cells within the scaffold (can be tested in the presence or absence of test compounds. In these embodiments, the second population of cells can be used to recreate different tissue-specific cellular niches.

### EXAMPLES

### Chemical and Physical Characterization of Human Plasma 3D Culture Model:

Methods: 3DeTME cultures are formed through the cross-linking of fibrinogen found naturally in plasma. Cross-linking time was assessed by measuring the time necessary to achieve matrix cross-linking using three relevant cross-linkers of the blood coagulation process including Thrombin (0-5 mg/ml), CaCl2 (0-5 mg/ml), and Factor XIII (0-6 mg/ml). The stabilization effects of preventing fibrin degradation and stability improvement in the scaffold was assessed by surveying several chemical antifibrinolytic agents including tranexamic acid (AMCHA) (0-10 mg/ml), Aprotinin (0-550 mg/ml), epsilon-aminocaproic acid (EACA) (0-2.5 mg/ml), and 4-aminomethylbenzoic acid (PAMBA) (0-2.5 mg/ml). The stability of the scaffold was studied by measuring each scaffold weight at day 0 and again measuring scaffold weight at the conclusion of a 3 week time period. 3DeTME culture scaffold structure and morphology was analyzed with scanning electron microscopy (SEM) using a FEI Quanta™ 450 Scanning Electron Microscope at multiple magnifications. The stiffness of the scaffolds was measured by atomic force microscopy (AFM). The Young's modulus was estimated by fitting a modified Hertz model onto the AFM indentation curve using the built in function of AFM software (Asylum Research). Plasma from cancer patients and healthy subjects was analyzed for fibrinogen content through the clotting method of Clauss. The Clauss fibrinogen assay is a quantitative, clot-based, functional assay. The assay measures the ability of fibrinogen to form fibrin clot after being exposed to a high concentration of purified thrombin. Briefly, plasma samples were loaded into the STA-R™ Evolution Expert Series Hemostasis System (Diagnostica Stago Inc., Parsippany, N.J.) and automated testing was carried out by the analyzer. Control reagents were prepared and run to confirm accurate and reproducible results. The effect of cytokines contributed by healthy and cancerous plasma used in the 3DeTME model was tested using a custom cytokine antibody array. Acellular 3DeTME cultures were created with either plasma from a healthy subject or plasma from a cancer patient using serum-free media. After chemical cross-linking and stabilization was complete, the cultures were disrupted with a lysis buffer (created by combining RIPA buffer, PMSF (1:10), DMOG (1:10), DTT (1:5), phosphatase cocktail 2 and 3 (1:100)) and sonication. 3DeTME culture supernatants were collected and analyzed by a C-Series Custom Cytokine Antibody Array (RayBiotech Inc., Norcross, Ga.) according to the instructions provided by the manufacturer. The custom cytokine array includes the following cytokines: interleukin beta 1 (IL-β1), macrophage inflammatory protein 1 alpha (MIP-1a), epidermal growth factor (EGF), insulin-like growth factor 1 (IGF-1), hepatocyte growth factor (HGF), platelet-derived growth factor AB (PDGF-AB), interferon gamma (INF-γ), interleukin-2 (IL-2), tissue inhibitor of metalloproteinase (TIMP), and matrix metallopeptidase (MMP). Images of the chemiluminescence signals of each of the membranes were captured using a LI-COR Odyssey™ (LI-COR Biosciences, Lincoln, Nebr.) device with a 2 minute exposure time. The chemiluminescence signal intensity of each spot was quantified by densitometric analysis (VisionWorks Software). Values for each cytokine were established by initially subtracting negative controls and then normalizing to positive controls for each of the membranes.

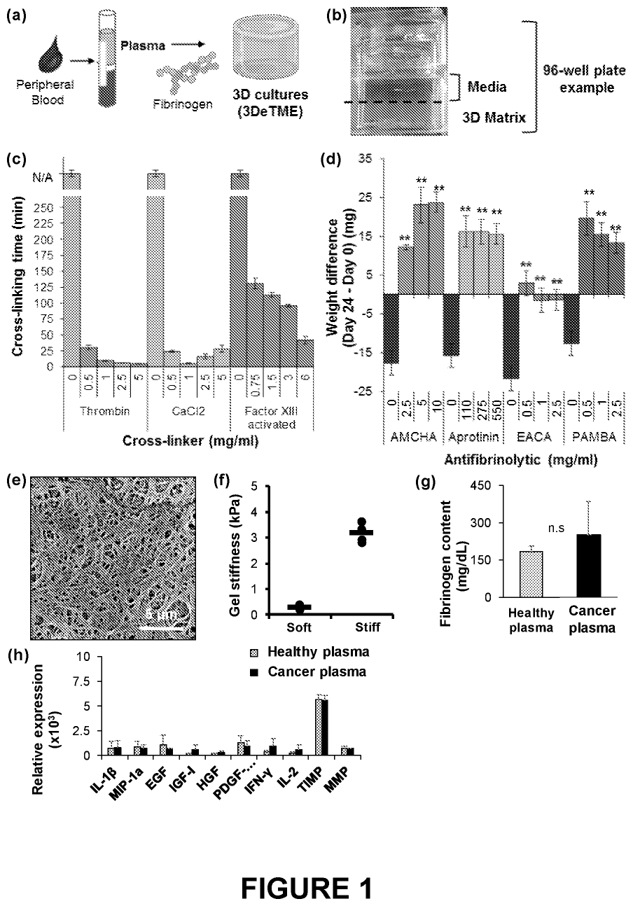

Results: Human plasma-derived 3D culture (3DeTME) models were created by cross-linking fibrinogen; a blood plasma protein responsible for normal blood clotting when converted into fibrin (FIG. 1a), generating a gelatinous-like scaffold matrix using traditional tissue culture surfaces as the recipient mold, with media added on top to overcome drying of the matrix (FIG. 1b). To optimize conditions for cell culture, we needed a stable 3D matrix with fast, yet controlled cross-linking capabilities and a porous intrinsic structure. For that purpose, three classical cross-linkers were tested to determine which component would produce optimal cross-linking of the 3DeTME. Plasma requires the presence of a cross-linking agent in order to form a 3D scaffold matrix, otherwise it remains in a liquid form with no reportable cross-linking time (represented as not applicable, N/A) when no cross-linking agent is added. The addition of thrombin allowed the cross-linking time to be reduced with increasing concentrations to a value of 5 min at 5 mg/ml. Adding CaCl₂ generated the fastest cross-linking time (4 min) at a concentration of 1 mg/ml, and increasing concentrations proved to be less efficacious. Factor XIII required activation by incorporating calcium, and the fastest cross-linking time for this component was over 40 min at a concentration of 6 mg/ml (FIG. 1c). With this data, CaCl₂ at a 1 mg/m1 concentration was determined to be the optimal concentration for cross-linking for the remainder of the experiments. Another important aspect to consider is that fibrin clots tend to degrade or lyse overtime so, in order to reduce 3DeTME degradation, as well as maintain structural integrity and stability, various antifibrinolytics were tested. 3DeTME integrity and stability was measured after 24 days in culture by comparing the weight of the 3D cultures at day 24 to the weight of the 3D cultures at day 0. A lack of antifibrinolytics incorporated into the matrix resulted in a weight loss of about 16.98±3.77 mg (representing around 5 to 9% loss of total weight). While, epsilon-aminocaproic acid (EACA) was not able to sustain an integrity benefit at the concentrations tested, the other 3 antifibrinolytics resulted in weight gains of at least 5 to 10% of their total weight (FIG. 1d). In particular, trans-4-(aminomethyl)cyclohexanecarboxylic acid (tranexamic acid) at 5 and 10 mg/ml resulted in the highest weight gain of about 24 mg (representing around 10% gain of total weight) and this was defined to be the recommended concentration for all remaining experiments. Working under the recommended cross-linker and stabilizer concentrations, scanning electron microscopy (SEM) was used to determine the physical structure of 3DeTME cultures. SEM (FIG. 1e) images revealed a porous structure with a network of interconnecting fibers, which will aid in gas diffusion, nutrient supply, and waste removal through the 3D culture matrix. Scaffold stiffness revealed soft and stiff tissue-like values of about 0.5 and 3 kPa, respectively (FIG. 1f). Fibrinogen levels were found to be non-significantly different between plasma from healthy subjects and cancer patients (FIG. 1g). In addition, plasma, from healthy subjects and cancer patients, was cross-linked to generate 3DeTME cultures, which were further characterized by the cytokine milieu of the 3D culture matrix. Using a custom antibody array, we measured proteins (in duplicate) at the baseline, day 0, of acellular 3DeTME cultures in serum free media. Relative protein expression was compared for healthy subjects and cancer patients and no significant differences were found (FIG. 1h).

Conclusions: 3DeTME scaffolds formed through the cross-linking of fibrinogen found, naturally in plasma, into fibrin by the mixture with crosslinkers and stabilizers generated a tissue-like environment with a porous intrinsic nature, tissue-like stiffness, and was found to be a good reservoir for fibrinogen and cytokines (pro-inflammatory cytokines, cytokines involved in fibrogenesis, cytokines supporting tissue repair/extracellular matrix degradation and remodeling, and cytokines promoting cell growth). The similarly in fibrinogen and cytokine content found in healthy and cancer plasma allows for a more relevant comparison among the 3DeTME cultures using either healthy or cancer patient plasma. 3DeTME scaffolds are patient-derived and do not include exogenous components.

### 3DeTME Culture Supports Cancer Proliferation:

Methods: Breast cancer cell lines (Luminal A: MCF7, ZR-75-1, HER2: MDA-MB-453, SK-BR-3 and Triple Negative: MDA-MB-231) were previously labeled with DiO and incorporated in 3DeTME alone or in co-culture with tumor microenvironment cellular component from primary tissue biopsies. These cultures were grown and analyzed at days 0.5 (γ0), 3, and 7. On each day of analysis, 3DeTME were enzymatically digested with type I collagenase at a concentration of 20 mg/m1 for 2-3 hours at 37° C. After 2-3 hours of incubation, samples were prepared in PBS for flow cytometry by adding counting beads (424902, Biolegend, CA) in addition to Sytox™ blue dead cell stain (excitation 358 nm; emission 461 nm) (S34857, Thermo Fisher Scientific, MA) for viability to each sample. BCa cells were identified by gating live cells with a DiO+ signal using the FITC channel on the BD FACS LSRFortessa™ SORP. A minimum of 5×10³ events was acquired per sample and the FACSDiva v.6.1.2 software was used to collect and interpret data. BCa cell counts were acquired and data was analyzed using FlowJo™ v10 (Ashland, Oreg.). Data was normalized to a predetermined number of counting beads and the proliferation of each condition (fold of γ0) was calculated and compared. These scaffolds were also fixed in 10% neutral buffered formalin and processed on a Leica 300 ASP tissue processor. Paraffin-embedded 3D matrix sections were longitudinally sliced at 10 μm. The BenchMark® XT automated slide staining system (Ventana Medical Systems, Inc., AZ) was used for antibody optimization and staining. The antigen retrieval step was performed using the Ventana CC1 solution, which is a basic pH Tris based buffer. Both primary and secondary antibodies were prepared in a 1× permeabilization buffer (BioLegend, CA). The Ventana iView™ DAB detection kit was used as the chromogen, and the slides were counterstained with hematoxylin. Anti-Ki-67 (CRM325, 1:100, Biocare Medical) and anti-cleaved caspase 3 (CRM229, 1:100, Biocare Medical) primary antibodies were used. The omission of the primary antibody served as negative control. Secondary antibodies used were biotin-conjugated goat anti-rabbit IgG (111-065-144, 1:1,000, Jackson ImmunoResearch, PA) and biotin-conjugated donkey anti-mouse IgG (715-065-151, 1:1,000, Jackson ImmunoResearch, PA), respectively. IHC images were imaged using an Aperio VERSA™0 Bright field Fluorescence & FISH Digital Pathology Scanner (Leica, N.J.). Growth and dissemination of cancer cells-DiO within the 3DeTME scaffolds was observed using confocal microscopy at day 3 and day 7. The 3DeTME structure was formed in an 8-well Thermo Scientific Nunc™ Lab-Tek™ II Chambered Coverglass with a No. 1.5 borosilicate glass bottom and covered with DMEM or RPMI-1640 media. The culture tray was imaged using a Nikon Ti2-A1TR™ confocal microscope with a 10× objective lens. Culture cells were exposed to 488 nm (DiO) excitation and the light emissions at 500-530 nm were collected as a z-stack image of each scaffold with a depth of roughly 0.5 mm to 1 mm using a step size of 2 μm. The frame size of the image was 512×512 pixels which was taken at a rate equivalent to 1 μs/pixel.

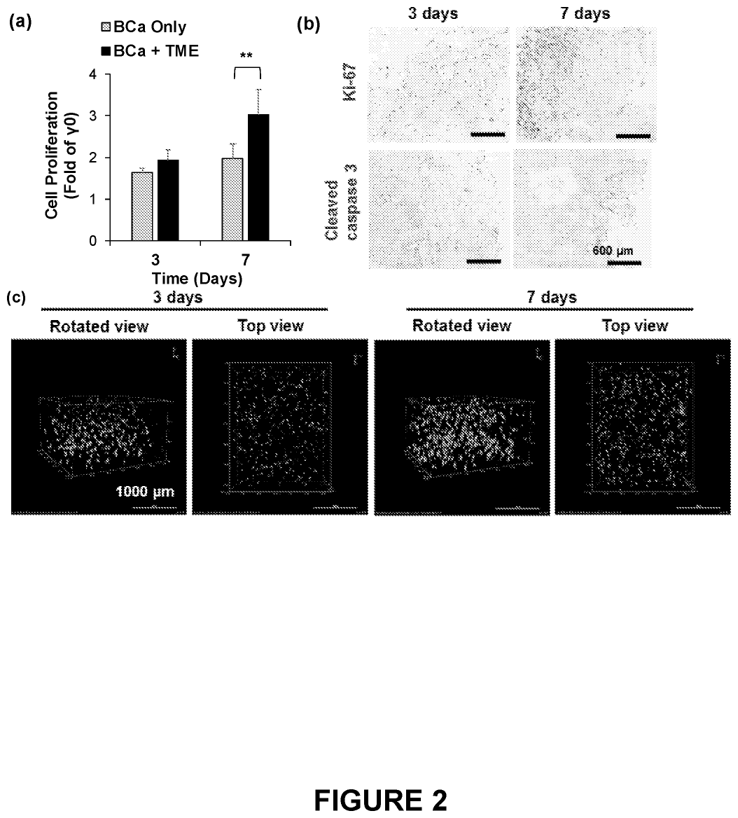

Results: The five BCa cell lines alone showed very similar results in proliferation with an increased proliferation of approximately 1.6-fold and 2-fold compared to γ₀ at day 3 and 7, respectively. However, co-culture with TME at day 7 significantly increased cell proliferation to 3-fold in all the BCa cell lines tested, reflecting the important role of the TME on tumor proliferation (FIG. 2a). We further confirmed these results by IHC, which revealed an increased proliferation through pixel count, of an increased Ki67 expression over time, at day 7, while apoptosis expression, measured by cleaved caspase 3, remains unaltered (FIG. 2b). Moreover, we evaluated 3DeTME by confocal imaging (FIG. 2c). 3DeTME revealed a significant increase in the number of BCa cells (DiO labeled) and increased clustering capabilities at day 7 compared to day 3. Confocal imaging revealed cell-to-cell and cell-to-matrix interactions relevant for recapitulation of key cellular interactions.

Conclusions: 3DeTME supports the efficient growth and expansion of cancer cells with increased proliferation overtime while 110 cell apoptosis by allowing cellular interactions in a tissue-like 3D architecture.

### 3DeTME Culture Allows High-Throughput Drug Screening:

Methods: Three breast cancer (BCa) cell lines were previously labeled with DiD and incorporated in 3DeTME. Half a day after plating, cells were treated with a DMSO control (γCtrl) and increasing concentrations 0.1 nM-300 μM of seven standard-of-care chemotherapeutic drugs including Methotrexate (MTX), Paclitaxel (PTX), Capecitabine (CAP), Cyclophosphamide Monohydrate (CYCLO), Carboplatin (CARBO), Epirubicin Hydrochloride (EPI), and Docetaxel (DTX). Treatments were added on top of 3DeTME in order to simulate drug diffusion into a tumor. Treatments were refreshed at day 4 and BCa cells were retrieved from the different cultures for analysis at day 0.5 (γ0) and day 7. Samples were prepared in PBS for flow cytometry by adding counting beads (424902, Biolegend, CA) in addition to Sytox™ green dead cell stain (excitation 504 nm, emission 523 nm) (S7020, Thermo Fisher Scientific, MA) for viability to each sample. BCa cells were identified by gating live cells with a DiD+ signal using the FL4 channel on BD Accuri™ C6 instrument (CFlow Software) (BD Biosciences). A minimum of 5×103 events was acquired per sample and BCa cell counts were acquired and data was analyzed using the FlowJo™ v10 (Ashland, Oreg.) software. Relative cell count and Growth rate (GR) values. The GR values show the partial inhibition effect of the drug when it achieves GR values from 0 to 1, with the cytostatic effect being represented when the value is equal to 0 and the cytotoxic effect being represented when it lies between 0 and −1.

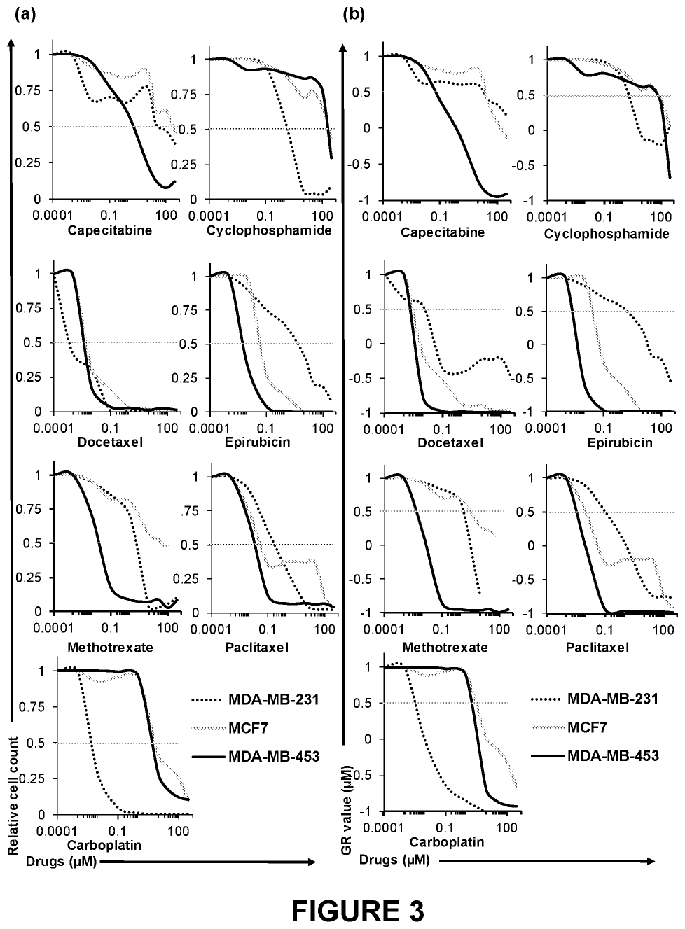

Results: Relative cell count (FIG. 3a) and GR value (FIG. 3b) curves for all the screened conditions revealed heterogeneous therapeutic responses. For example, methotrexate and carboplatin showed a heterogeneous response among the BCa cell lines with MDA-MB-231 being the most sensitive to carboplatin and MCF7 being the most resistant to methotrexate. Epirubicin metrics were consistent in cell count and GR curves which exhibited MDA-MB-231 as the most resistant cell line and capecitabine was revealed as a cytostatic drug over the concentrations tested.

Conclusions: 3DeTME allows high-throughput drug screening. While relative cell count considered the effect of the drug at the final time of the assay, GR parameters considered the initial cell population and the differences in the growth rates among the BCa cell lines in the 3DeTME. Our studies looked at whether differences in cell growth rates of cancer cells in the 3DeTME and a wide variety of drug metrics could radically impact drug responses, leading to an incomplete picture when predicting drug efficacies and provided drug response in a short time (7 days). We detected a significant heterogeneity among the different BCa cell lines, drugs and drug response metrics, suggesting the need for the use of more than one type of drug response metric to predict drug efficacy and the requirement of a method for personalized prediction of therapeutic response.

3DeTME Culture Drug Metrics Correlate Better than Other In Vitro Models with Clinical Data and Promote Growth of Patient Biopsy Material and Recreate Therapeutic Responses Shown in Patients:

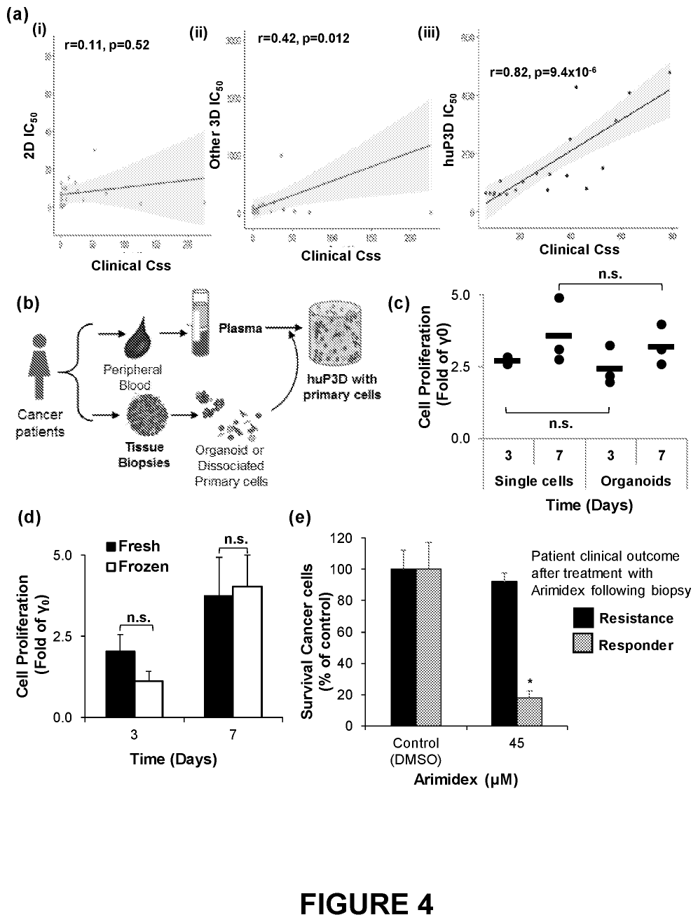

Methods: To evaluate the association between different variables, correlation tests were performed using the ggpubr R™ package. The Pearson correlation (r) was assessed in order to measure the linear dependence between two variables after confirmation of a normal distribution of the data. In order to assess the predictive value of the drug response metrics obtained in the 3DeTME assays with cell lines, we compared them with metric data obtained from literature relevant for 2D models (IC₅₀) and other 3D models (IC₅₀), as well as effective concentrations in patients from phase I or II studies that examined the pharmacokinetics of the tested chemotherapies (steady state concentration, Css). A scatterplot correlation graph allowed us to establish the strength, direction and form of the relationship between the in vitro models and the Css clinical data, with Pearson correlation coefficients (r) that were calculated to measure the strength of those relationships. Fresh or frozen small organoids and single cells obtained from BCa patient biopsies were incorporated in 3DeTME cultures (FIG. 4b). Briefly, tissues were weighed, pre-washed and minced into pieces approximately 0.2 mm² with a sterile scalpel and forceps. Minced tissue biopsies were enzymatically dissociated in dissociation buffer (0.1% W/V type I collagenase and 3 mM CaCl₂ solution), using a guideline of 1 ml dissociation buffer per 100 mg tissue, followed by sequential filtration for the generation of small organoids and single cell suspensions. Small organoids and single cells cultures were grown and analyzed at days 0.5 (γ₀), 3, and 7. Breast cancer patients with a known clinical outcome and treated with the same chemotherapeutic regimen were identified. Patient clinical follow-up was greater than two years and their response was categorized as resistance or response to treatment. These cultures were grown and treated with a DMSO control (γ_{Ctrl}) and Arimidex concentration of and 45 μM (Css). Treatments were refreshed at day 4 and BCa cells were retrieved from the different cultures at day 7. On each day of analysis, 3DeTME cultures were enzymatically digested and isolated cells were stained with FITC conjugated anti-CD45 (304038, Biolegend, CA), BV605 conjugated anti-CD44 (103047, Biolegend, CA), and PECy7 conjugated anti-EpCAM CD326 (324222, Biolegend, CA). Samples were prepared in PBS with 1% BSA (W/V %, Sigma-Aldrich, Saint Louis, Mo.) for flow cytometry by adding counting beads (424902, Biolegend, CA) in addition to Live/Dead Blue Cell Stain™ (L34962, Thermo Fisher Scientific, MA) for viability to each sample. BCa cells were identified by gating live cells as CD45−/CD44+/EpCAM+ cells on the BD FACS LSRFortessa™ SORP. A minimum of 5×10³ events was acquired per sample and FACSDiva™ v.6.1.2 software was used to collect data. BCa cell counts were acquired and data was analyzed using FlowJo™ v10 (Ashland, Oreg.). Data was normalized to a predetermined number of counting beads, the proliferation of each condition (fold of γ₀) and survival (% of control) was calculated and compared.

Results: While a very weak positive correlation (r=0.11) existed for the comparison of 2D IC₅₀ to clinical Css values (FIG. 4a(i)), moderate (r=0.42) to strong (r=0.82) correlations were revealed for IC₅₀ values of other 3D models (FIG. 4a(ii)) and the 3DeTME culture model (FIG. 4a(iii)) compared to the clinical Css, respectively. 3DeTMF primary cultures were developed using tissue biopsies and matching plasma from the same BCa patient (FIG. 4b). Cell proliferation of primary BCa cells by both methodologies (single cells and organoids) for the processing of fresh biopsies was not found to be significantly different with about a 2.5-fold and 3.3-fold growth compared to day 0 at days 3 and 7, respectively (FIG. 4c). We further compared the feasibility of growing the same biopsy directly from fresh tissue or after a freeze/thaw cycle using the single cell suspension methodology. Cell proliferation of BCa cells from frozen conditions remained unaltered when compared to the cells from fresh tissue with about a 2.3-fold and 3.7-fold growth compared to day 0 at days 3 and 7, respectively (FIG. 4d). Successful growth of frozen biopsies allowed us to further use frozen biopsies with a known clinical outcome after treatment with the same chemotherapeutic regimen. Plasma and biopsies from each of these patients was used in a precision-based approach and tested with the same chemotherapeutic regimen (Arimidex) as was utilized in the clinic after biopsy collection. Survival of BCa cells after Arimidex treatment correlated with the reported clinical outcome. While EpCAM+ BCa cells from patient with the “resistance” clinical outcome clearly revealed little to no effect of Arimidex at 45 μM, BCa cells decreased to 17% for a “responder” patient (FIG. 4e).

Conclusions: Our results showed the feasibility and efficacy of the 3DeTME drug response metrics to predict clinically effective therapies better than current preclinical models (2D and other 3D). 3DeTME cultures demonstrated two successful methodologies to grow primary patient material as well as confirming consistent growth from fresh or frozen biopsies. It is important to emphasize that primary BCa cultures in 3DeTME models contained BCa cells and all accessory TIVIE cellular components from the original biopsy, recapitulating the in vivo environment of the BCa cells in a 3D culture. Finally, we were able to retrospectively predict the same clinical outcomes detected in a clinical setting using primary biopsies included in 3DeTME and tested for the same drug regimen than in the clinic. These results highlighted the feasibility of the precision-based capabilities of 3DeTME cultures for the prediction of therapeutic efficacy.

### Development of 3DeTME Cultures for Recapitulation of Physiologically Relevant Oxygen and Tumor-Immune Interactions:

Methods: 3DeTME cultures were grown with cancer cells for 4 days, while being exposed to variable O₂ environments (21% and 1.5% O₂). Peripheral blood mononuclear cells (PBMCs) were incorporated at day 4 as a cell suspension in the medium added on the top of the matrix, while being exposed to the same O₂ environment up to day 7, (FIG. 5A). Oxygen partial pressure (pO₂) levels were measured in BCa cell-seeded 3DeTME matrices incubated under variable O₂ environments (21% and 1.5% O₂) after 0, 2, 4 and 7 days of culture. 3DeTME scaffolds containing BCa cells were profiled along the z-direction with an oxygen microsensor (Needle-Type Oxygen Microsensor NTH-PSt7, PreSens, Regensburg, Germany) and a manual micromanipulator (FIG. 5B). Briefly, to record oxygen pressure, the sensor was introduced into the geometric center (3 measure points) of the 3DeTME and moved from the border between the media and 3DeTME (top) in 10 μm steps towards the bottom of the well plate, as illustrated in FIG. 5C. The Software, PreSens Profiling Studio, enabled the measurement of variable step sizes, measuring velocities and wait times. Before application, a two-point calibration was performed: 1.5% O₂ in an enclosed chamber as 1.5% O₂ reference and ambient air as 21% O₂ reference.

Results: After 4 days in culture, cell-seeded 3DeTMF matrices incubated at 21% O₂ were found to exhibit a top pO₂. value of 7.3±1.3 kPa and a bottom value of 5.3±2.6 kPa (FIG. 5D). A gradual decrease in pO₂ levels was manipulated by oxygen incubation in a hypoxic globe chamber. When the scaffolds were incubated at 1.5% O₂ the pO₂ levels developed within the scaffolds dropped to 0.7 kPa at the top to 0.4 kPa in the bottom (FIG. 5D). Hereafter, the 3DeTME matrices will be referred to as 3DeTME physiological (reflecting an average pO₂ content of 6.3±2.1 kPa) and 3DeTME tumorous (reflecting an average pO₂ content of 0.64±0.08 kPa), respectively.

Conclusions: 3DeTME recapitulated key oxygen levels physiologically relevant to healthy and tumor tissue allowing us to explore further the role of oxygen availability in tumor biology and tumor-immune interactions.

### Characterization of 3DeTME Physiologically Relevant Oxygen Effect on Cell Biology:

Methods: 3DeTME matrices were enzymatically digested with collagenase (20 mg/ml for 2-3 hours at 37° C.) on day 4. BCa cells were isolated and identified by gating cells with a high DiO signal (excitation, 488 nm; emission, 530/30 nm). Antibody used to evaluate hypoxic status was AlexaFluor™ 647 conjugated anti-hypoxia inducible factor (HIF)1α (359706, Biolegend, CA). Cell viability was evaluated by using a Sytox™ Blue live-dead fluorescent dye (S34857, Invitrogen, CA) possessing excitation, 358 nm; emission, 461 nm or Live/Dead Blue cell stain (L34962, Thermo Fischer Scientific, MA). For all analyses, a minimum of 5,000 events were acquired using BD FACS Fortessa™ and FACSDiva™ v6.1.2 software or BD FACS Accuri and BS Accuri™ C6 software (BD Biosciences), respectively. The BCa cell counts were always normalized to a predetermined number of counting beads (424902, Biolegend, CA), and mean fluorescence intensity (MFI) was assessed with respect to the corresponding isotype in the BCa-DiO+ cells. The data was analyzed using FlowJo™ program v10 (Ashland, Oreg.). Paraffin section cuts of 3DeTME matrices were imaged using a Nikon Ti2-A1TR™ confocal microscope (×20 dry, ×40 oil and ×60 oil objectives, 2.5 magnified) and analyzed using NIS elements software (Nikon, Melville, N.Y., USA). For IF studies, paraffin sections were dewaxed in the following order: 10 minutes in xylene, 10 minutes in 100% ethanol, 10 minutes in 95% ethanol, 10 minutes in 70% ethanol and 10 minutes in distilled water, followed by rehydration in wash buffer (0.02% BSA in PBS) for 10 minutes. After this, sections were subjected to incubation in blocking buffer (5% BSA in PBS) for 60 minutes at room temperature to block non-specific staining between the primary antibodies and the sample. Sections were rinsed with washing buffer and incubated in incubation buffer (1% BSA in PBS) with different primary antibodies. Primary antibody incubation was carried out overnight at 4° C. to allow for the optimal binding of antibodies to sample targets and reduce non-specific background staining. Anti-collagen-I (MA1-26771, 1:100, Thermo Fischer Scientific, MA), anti-collagen-III (SAB4200749, 1:100, Sigma Aldrich, MO), and an AlexaFluor™ 647 conjugated anti-HIF-1α were used (359706, 1:100, Biolegend, CA). A FITC conjugated secondary antibody (SAB4600042, 1:1000, Sigma Aldrich, MO) was used whenever applicable. For samples stained with anti-HIF-1α, blocking and incubation buffers were prepared in 1× permeabilization buffer (Biolegend, CA). The dilution of antibodies was carried out according to the manufacturer's instructions. Lastly, a drop of anti-fade mounting media containing DAPI was added to the samples and sections were imaged.

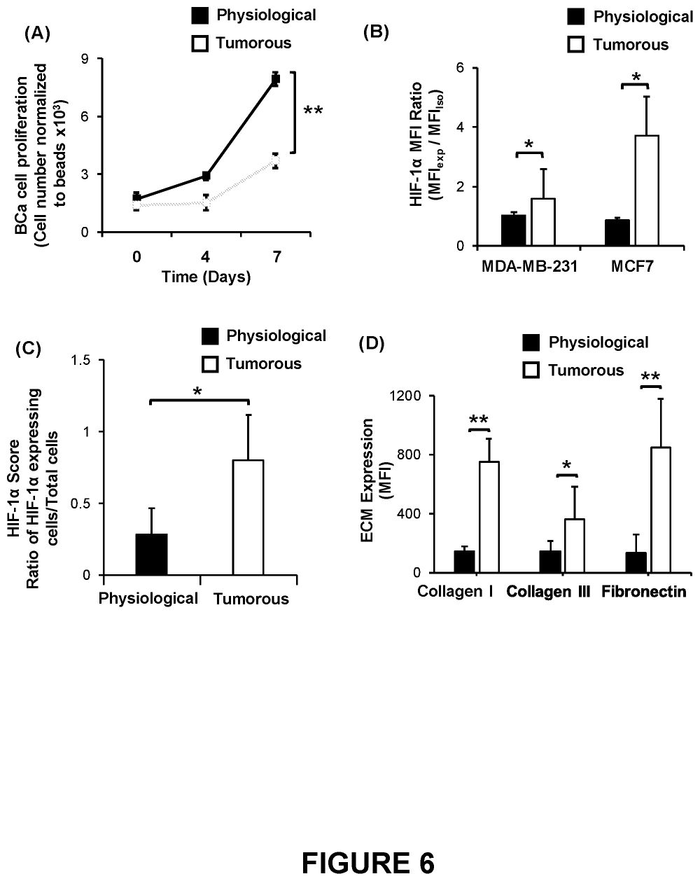

Results: To evaluate the impact of an oxygen-deprived environment on BCa proliferation, we analyzed BCa cell numbers in 3DeTME physiological and tumorous matrices by flow cytometry. As illustrated in FIG. 6A, the rate of BCa cell proliferation was observed to be significantly hindered in 3DeTME tumorous compared to 3DeTME physiological model at days 4 and 7. 3DeTME tumorous matrices showed a significant increase in the number of BCa cells expressing HIF-1α, in which the HIF-1α MFI ratio was 1.6 and 3.7 times higher compared to 3DeTME physiological matrices for MDA-MD-231 and MCF-7, respectively (FIG. 6B). We further corroborate these findings using paraffin section of 3DeTME cultures. Immunofluorescence of 3DeTME physiological and 3DeTME tumorous scaffold sections using anti-HIF-1α antibody revealed that the HIF-1α, score (ratio of positive HIF-1α expressing cells/total cells) was significantly higher for BCa cells grown under oxygen-deprived conditions in 3DeTME tumorous scaffolds compared to the BCa cells grown in 3DeTME physiological scaffolds, as illustrated in FIG. 6C. To characterize the role of oxygen deprivation in the surrounding matrix, we studied the expression of main fibrous extracellular matrix (ECM) proteins in breast tissue including collagen I, collagen III and fibronectin under 3DeTME tumorous and physiological conditions. Quantification of these fibrous ECM proteins indicated a significantly increased expression (FIG. 6D).

Conclusions: 3DeTME cultures mimic oxygen availability relevant to healthy tissue and blood physiological levels that circulating and immune cells are exposed to, as well as, pathophysiological oxygen levels occurring in tumor tissue. Our results confirm that oxygen deprivation within 3DeTME matrices can efficiently reiterate HIF-driven regulation in the resident BCa cells by decreasing cell proliferation, upregulating HIF-expression and ECM remodeling with increased ECM deposition, known intratumoral hypoxic hallmarks.

### Characterization of 3DeTME Physiologically Relevant Oxygen Effect on Tumor Immune Interactions and Drug Response to Immunotherapy:

Methods: Differences in lymphocyte infiltration into 3DeTME scaffolds as a result of different oxygen content were assessed. PBMCs were incorporated as cell suspension in the medium added on the top of the matrix at day 4 and analyzed at day 7. 3DeTME matrices were enzymatically digested with collagenase and PBMCs were isolated and surface-stained with the following antibodies: FITC conjugated anti-CD3 (300406, Biolegend, CA), PE-Cy5 conjugated anti-CD4 (300508, Biolegend, CA), APC-Cy7 conjugated anti-CD8 (344714, Biolegend, CA), APC conjugated anti-CD19 (302212, Biolegend, CA) and BV510 conjugated anti-CD45 (304036, Biolegend, CA). Infiltrated populations were characterized with manual gating, or combined datasets were down-sampled and subjected to dimensionality reduction using t-stochastic neighbor embedding (t-SNE) algorithm (Abdelmoula et al. 2016) or automatically defined with FlowSOM™ clustering algorithm (Potts et al. 2007). Mean absolute numbers of CD3+, CD4+, CD8+ and CD19+ cells (normalized to beads) were determined in each experimental group. To confirm differences in CD8+ infiltration as a result of oxygen content variations, the number of infiltrated CD8+ cells were imaged using confocal microscopy or IHC. We further examined a selective, high-affinity human IgG1 mAb that blocks programmed cell death ligand-1 (PD-L1) binding to PD-1 (Durvalumab, 5 μM) to evaluate its role on CD8 infiltration by flow cytometry.

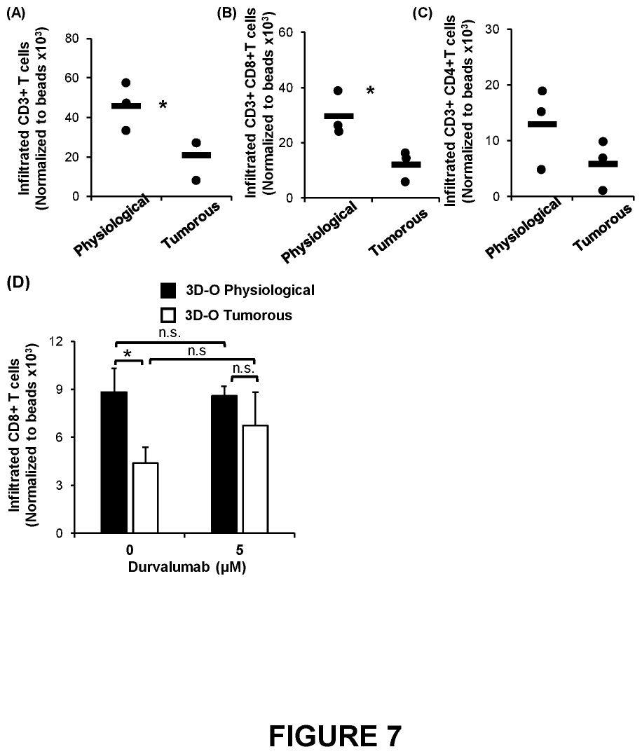

Results: We identified significantly impaired infiltration of CD3+ (FIG. 7A), CD8+ (FIG. 7B), and reduced CD4+ cells (FIG. 7C) inside 3DeTME tumorous compared to 3DeTME physiological. Additionally, we found that treatment with an investigational anti-PD-L1 monoclonal antibody (Durvalumab, 5 μM) did reverse CD8 infiltration in 3DeTME tumorous to the cell infiltration numbers of the 3DeTME physiological matrices.

Conclusions: We have demonstrated that 3DeTME recapitulates tumor-immune interactions and BCa cells grown within the oxygen deficient-niche of 3DeTME tumorous scaffolds could promote tumor-immune evasive events. CD3+ and CD8+ T cells infiltration was significantly impaired under pathophysiological oxygen levels in the 3DeTME tumorous model. PD-L1 inhibition re-sensitized BCa cells to cytotoxic CD8+ T cell infiltration showing the capabilities of 3DeTME to assess treatment strategies for hypoxia-modification therapy and to reverse immune evasion.

1. A method, comprising:
(a) mixing peripheral blood plasma, with cross-linker and stabilizer to form a mixture; and
(b) incubating the mixture for a time and under conditions to form a three-dimensional cross-linked scaffold.

2.-18. (canceled)

19. A three-dimensional cross-linked scaffold comprising peripheral blood plasma.

20. The three-dimensional cross-linked scaffold of claim 19, wherein the scaffold further comprises biological cells, such as human cells, within the scaffold.

21.-22. (canceled)

23. The three-dimensional cross-linked scaffold of claim 20, wherein the biological cells are present in the scaffold at a concentration between about 10³ cells/ml and about 10⁷ cells/ml, between about 10³ and about 10⁶ cells/ml, between about 10⁴ and about 10⁷ cells/ml, between about 10⁴ and about 10⁶ cells/ml, between about 10³ and about 10⁵ cells/ml, or between about 10⁵ and about 10⁷ cells/ml.

24. The three-dimensional cross-linked scaffold of claim 19, comprising a cross-linker selected from the group consisting of calcium chloride, thrombin, and factor XIII, or a combination thereof.

25. The three-dimensional cross-linked scaffold of claim 24, comprising (i) calcium chloride present at a concentration of between about 0.5 mg/ml and about 5 mg/ml, between about 0.5 mg/ml and about 4.5 mg/ml, between about 0.5 mg/ml and about 4 mg/ml, between about 0.5 mg/ml and about 3.5 mg/ml, between about 0.5 mg/ml and about 3 mg/ml, or between about 0.5 mg/ml and about 2.5 mg/ml; (ii) thrombin present at a concentration of between about 0.5 mg/ml and about 5 mg/ml, between about 1 mg/ml and about 5 mg/ml, between about 2 mg/ml and about 5 mg/ml, or between about 2.5 mg/ml and about 5 mg/ml, (iii) activated Factor III present at a concentration of between about 0.75 mg/ml and about 6 mg/ml, between about 1 mg/ml and about 6 mg/ml, between about 1.5 mg/ml and about 6 mg/ml, between about 2 mg/ml and about 6 mg/ml, between about 2.5 mg/ml and about 6 mg/ml, or between about 3 mg/ml and about 6 mg/ml; (iv) or mixtures thereof.

26. The three-dimensional cross-linked scaffold of claim 19, further comprising a stabilizer is selected from the group consisting of tranexamic acid, aprotinin, epsilon-aminocaproic acid and aminomethylbenzoic acid, or combinations thereof.

27. The three-dimensional cross-linked scaffold of claim 26, wherein the stabilizer comprises (i) tranexamic acid present at a concentration of between about 0.5 mg/ml and about 10 mg/ml, between about 1 mg/ml and about 10 mg/ml , between about 2 mg/ml and about 10 mg/ml, between about 2.5 mg/ml and about 10 mg/ml, between about 3 mg/ml and about 10 mg/ml, between about 3.5 mg/ml and about 10 mg/ml, between about 4 mg/ml and about 10 mg/ml, between about 4.5 mg/ml and about 10 mg/ml, or between about 5 mg/ml and about 10 mg/ml; (ii) aprotinin present at a concentration of between about 50 mg/ml and about 550 mg/ml, between about 75 mg/ml and about 550 mg/ml, between about 95 mg/ml and about 550 mg/ml, or between about 110 mg/ml and about 550 mg/ml; (iii) epsilon-aminocaproic acid at a concentration of between about 0.5 mg/ml and about 2.5 mg/ml, between about 0.5 mg/ml and about 2 mg/ml, between about 0.5 mg/ml and about 1.5 mg/ml, between about 0.5 mg/ml and about 1 mg/ml, or between about 0.5 mg/ml and about 0.5 mg/ml; (iv) aminomethylbenzoic acid at a concentration of between about 0.5 mg/ml and about 2.5 mg/ml, between about 0.5 mg/ml and about 2 mg/ml, between about 0.5 mg/ml and about 1.5 mg/ml, between about 0.5 mg/ml and about 1 mg/ml, or (v) combinations thereof.

28. The three-dimensional cross-linked scaffold of claim 19, comprising
(I) (A) calcium chloride present at a concentration of between about 0.5 mg/ml and about 5 mg/ml, between about 0.5 mg/ml and about 4.5 mg/ml, between about 0.5 mg/ml and about 4 mg/ml, between about 0.5 mg/ml and about 3.5 mg/ml, between about 0.5 mg/ml and about 3 mg/ml, or between about 0.5 mg/ml and about 2.5 mg/ml; and (B) tranexamic acid present at a concentration of between about 0.5 mg/ml and about 10 mg/ml, between about 1 mg/ml and about 10 mg/ml , between about 2 mg/ml and about 10 mg/ml, between about 2.5 mg/ml and about 10 mg/ml, between about 3 mg/ml and about 10 mg/ml, between about 3.5 mg/ml and about 10 mg/ml, between about 4 mg/ml and about 10 mg/ml, between about 4.5 mg/ml and about 10 mg/ml, or between about 5 mg/ml and about 10 mg/ml; or
(II) (A) comprising calcium chloride present at a concentration of between about 0.5 mg/ml and about 2.5 mg/ml; and (B) tranexamic acid present at a concentration of between about 5 mg/ml and about 10 mg/ml.

29. (canceled)

30. The three-dimensional cross-linked scaffold of claim 20, wherein (a) the biological cells are present at between about 10⁴ and about 10⁷ cells/ml or between about 10⁵ and about 10⁷ cells/ml, (b) no exogenous polymer is present in the three-dimensional cross-linked scaffold, and/or (c) wherein the peripheral blood plasma is present in the mixture at a concentration of between about 30% v/v and about 80% v/v, about 30% v/v and about 70% v/v, about 30% v/v and about 60% v/v, or between about 30% v/v and about 50% v/v, (d).

31.-32. (canceled)

33. The three-dimensional cross-linked scaffold of claim 19, wherein the scaffold has a thickness of 100 μm and about 3000 μm, between about 100 μm and about 2500 μm, between about 100 μm and about 2000 μm, between about 100 μm and about 1500 μm, between about 100 μm and about 1000 μm, between about 100 μm and about 900 μm, between about 100 μm and about 800 μm, between about 100 μm and about 700 μm, between about 100 μm and about 600 μm, between about 100 μm and about 500 μm, between about 100 μm and about 400 μm, between about 200 μm and about 1000 μm, between about 200 μm and about 900 μm, between about 200 μm and about 800 μm, between about 200 μm and about 700 μm, between about 200 μm and about 600 μm, between about 200 μm and about 500 μm or between about 200 μm and about 400 μm.

34. The three-dimensional cross-linked scaffold of claim 19, wherein the scaffold has an oxygen gradient.

35. The three-dimensional cross-linked scaffold of claim 19, wherein the scaffold comprises an oxygen partial pressure (pO₂) level between about 8.6 kPa and about 1.4 kPa.

36. The three-dimensional cross-linked scaffold of claim 35, wherein the scaffold comprises non-tumor biological cells, and wherein the pO₂ level is between about 8.6 kPa and about 2.5 kPa, between about 8.6 kPa and about 3.5 kPa, between about 8.6 kPa and about 4.5 kPa, between about 8.6 kPa and about 5.3 kPa, between about 8.6 kPa and about 5.9 kPa, or between about 7.3 kPa and about 5.3 kPa.

37. The three-dimensional cross-linked scaffold of claim 35, wherein the scaffold comprises tumor cells, and wherein the pO₂ level is between about 1.5 kPa and about 0.2 kPa, between about 1.5 kPa and about 0.3 kPa, between about 1.5 kPa and about 0.7 kPa, between about 1.2 kPa and about 0.2 kPa, between about 1.2 kPa and about 0.3 kPa, between about 1.2 kPa and about 0.7 kPa, or between about 0.7 kPa and about 0.3 kPa.

38. The three-dimensional cross-linked scaffold of claim 19, wherein the scaffold has a stiffness between about 0.5 kPa to 7 kPa,

39. The three-dimensional cross-linked scaffold of claim 38, wherein the scaffold comprises non-tumor biological cells, and wherein the stiffness level is between about 0.5 kPa to about 7 kPa, between about 0.5 kPa to about 6 kPa, between about 0.5 kPa to about 5 kPa, between about 0.5 kPa to about 4 kPa, between about 0.5 kPa to about 3 kPa, or between about 0.5 kPa to about 2 kPa.

40. The three-dimensional cross-linked scaffold of claim 38, wherein the scaffold comprises tumor cells, and wherein the stiffness level is between about 0.5 kPa to about 7 kPa, between about 1 kPa to about 6 kPa, between about 1 kPa to about 5 kPa, between about 1 kPa to about 4 kPa, or between about 2 kPa to about 4 kPa, or between about 0.5 kPa to about 2 kPa.

41. The three-dimensional cross-linked scaffold of claim 19, wherein the scaffold has a porosity is between about 0.5 μm and about 20 μm, between about 1 μm and about 15 μm, between about 1.5 μm and about 10 μm, or between about 2 μm and about 8 μm in diameter.

42. Use of the three-dimensional cross-linked scaffold of claim 19 for any suitable purpose, including but not limited to drug screening, tissue engineering, subject prognosis, cell metabolism, tumor heterogeneity, drug resistance studies, immune and oncology profiling, cell differentiation, toxicology studies, cell fate studies based on exposure to stimuli, inherent cell abnormalities, regenerative medicine, etc.

43. (canceled)
